(19) United States
(12) Patent Application Publication
(10) Pub. No.: US 2005/0272741 A1
(43) Pub. Date: Dec. 8, 2005

(54) Novel methods of treatment for Fontan patients with protein losing enteropathy

(76) Inventors: Jack Rychik, Merion, PA (US); Gil Wernovsky, Fort Washington, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/037,966

(22) Filed: Jan. 18, 2005

Related U.S. Application Data
(60) Provisional application No. 60/537,413, filed on Jan. 16, 2004.

(51) Int. Cl. A61K 31/519; A61K 31/401

(52) U.S. Cl. 514/252.16; 514/262.1; 514/423

(57) ABSTRACT

Methods for treating Fontan patients with protein losing enteropathy is provided.

### CROSS REFERENCE TO RELATED APPLCATION

This application claims priority to U.S. Provisional Application 60/537,413 filed Jan. 16, 2004, the entire disclosure of which is incorporated by reference herein.

### FIELD OF THE INVENTION

This invention relates to the fields of cardiology and pharmacology. More specifically, the invention provides methods for treating Fontan patients experiencing protein losing enteropathy.

### BACKGROUND OF THE INVENTION

Several publications and patents are cited in this application in order to more fully describe the state of the art to which this invention pertains. The disclosure of each of these citations is incorporated by reference herein.

Francis Fontan performed the Fontan operation for the first time in 1968. The Fontan operation is a heart operation used to treat complex congenital heart defects (birth defects of the heart) such as tricuspid atresia, hypoplastic left heart syndrome (HLHS), pulmonary atresia and single ventricle.

Protein losing enteropathy (PLE) is a frequent, debilitating and often lethal complication of the Fontan operation for the single ventricle type of congenital heart disease (CHD). PLE has been associated with low cardiac output, which may be due to increased pulmonary vascular resistance (PVR) in this complex form of CHD. Mesenteric vascular resistance (MVR), is also elevated in Fontan patients and particularly in patients with PLE (1,2). In addition to being a possible marker for excessive systemic vascular resistance (SVR) and contributing to a low cardiac output, high MVR may play a direct role in the pathophysiology of PLE.

PLE is an enigmatic ailment seen in 5-10% of patients after the Fontan operation. This disease is characterized by increased intestinal permeability to protein. As the disease progresses, increasing losses lead to severe hypoproteinemia with loss of vascular oncotic pressure and the development of edema, ascites, and pleural/pericardial effusions. In addition to albumin loss, hypogammaglobulinemia and lymphopenia occur, with impairment of immune function (1). Mortality is high with only 50% survival at five years from time of diagnosis. There are no effective treatments for PLE. Often even cardiac transplantation fails to reverse the process.

The precise pathophysiological mechanism of PLE after Fontan operation is unknown. Although a hemodynamic abnormality is the likely cause, retrospective data suggest the absence of a simple relationship between the degree of elevation of systemic venous pressure after Fontan operation and the development of PLE.

It is estimated that at least 2000 patients will have Fontan operations in North America each year. Annually, 100 to 200 patients will go on to develop PLE. The life expectancy of patients with Fontan physiology is unknown since the earliest survivors of single ventricle heart disease are only now reaching the age of 20.

### SUMMARY OF THE INVENTION

In accordance with the present invention, a novel method is provided for improving cardiac performance in a pediatric patient in need thereof. In a preferred embodiment, the method involves treating the patient with Sildenafil or derivatives thereof. Other embodiments entail treating the pediatric patient with Vardenafil HCl or Tadalafil.

In yet another aspect of the invention, the pediatric patient is a Fontan patient. In still another embodiment of the invention, the patient has protein losing enteropathy (PLE), wherein the patient may be a Fontan patient. The method may further comprise assessing the patient for clinical improvement as demonstrated by at least one parameter selected from the group consisting of a decrease in alpha trypsin clearance, an increase in serum albumin, an increase in cardiac output, a decrease in MVR, and a reduction in circulating TNF-alpha levels.

In an alternative embodiment of the invention, a combination therapy method is provided with entails administration of an effective amount of an agent selected from the group consisting of sildenafil, vardenafil HCl, or tadalafil in combination with administration of an effective amount of an agent selected from the group consisting of statins, ACE inhibitors, lasix, digoxin and aldactone to the pediatric patient.

### DETAILED DESCRIPTION OF THE INVENTION

Although survival has improved in recent decades, CHD remains the leading cause of childhood deaths from birth defects. Children with single ventricle disease account for a large portion of these deaths. Sildenafil or Viagra® has a unique potential to improve the physiologic state of patients with PLE and potentially all patients with Fontan due to its various mechanisms of action. However, this indication for Viagra has much broader implications, not only to other CHD patients, but to a broad spectrum of critically ill patients of all ages. The phenomenon of elevated MVR in PLE may be similar to that seen acutely in circulatory shock or chronic congestive heart failure in which blood flow is diverted away from the mesenteric circulation in order to preserve flow to the vital organs. The demonstration of Viagra's ability to decrease MVR and improve intestinal perfusion could open the door to many uses ranging from the prevention of necrotizing entrocolitis in premature infants to the treatment of mesenteric ischemia due to atherosclerotic disease in geriatric patients. In accordance with the present invention, Viagra's tremendous potential for therapeutic benefit in a PLE population that is desperate for new therapies and in whom the risk-to-benefit ratio will be assessed.

Doppler ultrasound of the superior mesenteric artery (SMA) confirms the presence of elevated MVR in patients after Fontan operation in comparison to normal. Most striking was the finding of markedly elevated MVR in those patients with PLE (n=13) in comparison to the Fontan patients without PLE (n=27) and to the normal controls (n=25) (2). Elevated mesenteric vascular resistance (MVR) may result in significant intestinal hypoperfusion leading to the observed disruption of the intestinal mucosal barrier and subsequent protein leak into the gut lumen.

In an ongoing study, we have investigated the relationship between enteric protein loss, MVR, and the inflammatory marker TNF-alpha after Fontan operation in patients with and without PLE. Twenty-eight patients with a mean age of 11.2±2.3 (range 5-16) years have been analyzed thus far. Our preliminary data supports the assertion that elevated vascular resistance, diminished flow, and inflammation play a role in the mechanism of this disease.

Use of Sildenafil (Viagra®, Pfizer, NY, N.Y.) for the Treatment of PLE:

Fontan patients have a low cardiac output. This is due to decreased myocardial performance and increased total vascular resistance (TVR). Since, in the Fontan operation, the systemic and pulmonary vascular circuits are in series without interposition of a pumping ventricle, TVR is the sum of the SVR, the fixed resistance of the Fontan baffle, and the PVR. By lowering TVR cardiac output can be augmented. In patients with PLE, in particular, augmentation of cardiac output (such as by fenestration of the Fontan baffle or by atrial pacing) has been a very successful strategy, often resulting in PLE remission (1). Hence, maximal augmentation of cardiac output is an objective in patients following the Fontan operation.

Without being bound to theory, Viagra, compared to all other therapies, may specifically benefit patients with Fontan physiology through 4 distinct mechanisms.

1. Lowering SVR

Afterload reduction (lowering SVR) with drugs that inhibit the angiotensin converting enzyme (ACE inhibitors), is a strategy that is often used in Fontan patients. Multiple studies have demonstrated Viagra's ability to safely lower SVR with only mild, acceptable alterations in blood pressure (3).

2. Lowering PVR

Due to the passive nature of pulmonary blood flow in Fontan patients, lowering PVR can also dramatically improve cardiac output. Viagra's ability to decrease PVR in children with pulmonary hypertension has been described (3) and is also the subject of an ongoing clinical trials.

Buvinic (4) has established the importance of nitric oxide (NO) as an endogenous activator of intracellular cGMP. This mechanism has been demonstrated as important in relaxing vascular smooth muscle, causing mesenteric vasodilatation in rats. Viagra's very distal action in this pathway, directly increasing cGMP, has a very potent ability to cause mesenteric relaxation. Following pretreatment with Viagra, even high dose Norepinephrine fails to constrict the mesenteric bed. This is vastly out of proportion to Viagra's effect on SVR in general. Preferentially decreasing MVR provides the other two mechanisms by which Viagra may improve Fontan physiology:

3. Increasing Intestinal Perfusion

Preventing hypoperfusion of the intestines may preserve the integrity of the mucosa to serum proteins and directly alter the course of PLE.

4. Suppression of Tumor Necrosis Factor (TNF)-Alpha Expression

Decreased sheer forces in the mesenteric vasculature may alter NO expression and contribute to TNF-alpha expression. TNF-alpha not only inhibits NO expression, but also acts as directly as a myocardial depressant (5). This myocardial depressive effect can further decrease cardiac output and may cyclically contribute to the downward spiral of pathophysiology seen in PLE patients. Viagra has the physiologic potential to suppress TNF-alpha expression at this level and break this cycle.

While Viagra is exemplified herein, other agents having a similar mechanism of action may also be employed. Exemplary agents include without limitation, vardenafil HCl available from Bayer Pharmaceuticals and tadalafil available from Eli Lilly and Co.

The following example is provided to illustrate an embodiment of the invention. It is not intended to limit the scope of the invention in any way.

### EXAMPLE I

Initially Viagra will be administered to Fontan patients with PLE. This subpopulation of the much larger Fontan population can be ideal candidates for this type of study due to the multiple levels of potential drug effect, the extremely large potential benefit, and the very high morbidity and mortality of PLE with currently available therapies. However, if overall cardiac performance is improved with Viagra, then all patients with Fontan physiology may benefit.

Methodology

The efficacy of Viagra for the treatment of Fontan patients with PLE can be assessed as follows. A randomized, prospective, double-blind, placebo cross-over study can be performed. Patients should be present for 4 separate days of testing. On each day the patient may provide a 24 hour stool sample for alpha-1-antitrypsin clearance as well as a blood sample for alpha-1-antitrypsin, complete chemistry (including total protein and albumin), TNF-alpha level, and Viagra level. The subject may also be interviewed and examined. On Day 1, a baseline echo can be obtained with measures of valvar regurgitation, ventricular function, cardiac output and MVR. A repeat echocardiogram can be obtained following IV Viagra/placebo. The patient is then discharged on oral Viagra/placebo for 6 weeks. Upon return (Day 2 of testing), repeat specimens may be collected and a repeat echocardiogram can be obtained. The oral therapy/placebo may be discontinued at the conclusion of this second visit. Following a 1 month washout period, the patient may return for the cross-over arm of the trial (Days 3 and 4).

Endpoints

Primary:

Clinical improvement can be defined by, without limitation:
1.20% decrease in alpha-1-antitrypsin clearance (the accepted standard for quantification of intestinal protein loss in PLE); and/or
2. 20% rise in serum albumin
   Secondary:
1.20% increase in cardiac output by Doppler echocardiography
2. 20% decrease in MVR
3. Decrease in serum TNF-alpha to normal
4. Decrease in reported symptoms of PLE and improvement in subjective assessment of health

### Treatment Including Dosing Information

Day 1 or 3: IV test dose of Viagra 0.1 mg/kg over 10 minutes. 15 minutes later a second dose of 0.25 mg/kg over 10 minutes may be administered. Blood pressures can be obtained every 2 minutes during infusion and every 5 minutes during the first 30 minutes following infusion. Discharge with stable vital signs 1.5 hours following end of the second infusion with TID oral dosing of Viagra based on body weight to begin the following morning and continue through day 2 or 4 of testing (approximately 6 weeks).

| Body Weight (kg) | Dose (mg) | Total daily dose (mg) |
|---|---|---|
| >8-20 | 20 | 60 |
| >20-45 | 40 | 120 |
| >45 | 80 | 240 |

Number of Subjects
20
Study Population
   Eligibility:
   1. Fontan physiology
   2. Age≧4
   3. Symptoms of PLE≧1 month
   4. Alpha-1-antitrypsin clearance>27 ml/24 hours
   5. Serum protein or serum albumin less than normal for age
      Exclusion Criteria:
   1. Presence of other sources of protein loss (e.g. nephrotic syndrome, Crohn's Disease)
   2. Pregnancy
   3. Hepatic or renal insufficiency
   4. Use of an alpha blocking medication in the month prior to enrollment
   5. Use of a nitrate based medication in the month prior to enrollment
   6. Use of another highly protein bound medication (e.g. warfarin) if the patient cannot be adequately monitored by his or her own physician for the activity of that medication while on this Viagra protocol

The present invention also provides a combination therapy method for treatment of the Fontan patient. An exemplary method entails the administration of an agent selected from the group consisting of sildenafil, vardenafil HCl, or tadalafil in combination with administration of an effective amount of an agent selected from the group consisting of statins, ACE inhibitors, lasix i.e., furosemide, digoxin i.e., digitalis and aldactone i.e., spironolactone to the pediatric patient. Exemplary statins include without limitation Lipitor and Crestor. An exemplary ACE inhibitor is Vasotec (Enalapril). The person skilled in the art of treating the pediatric Fontan patient can readily discern the appropriate dosages of the foregoing agents based on the age, weight and condition of the patient.

While certain of the preferred embodiments of the present invention have been described and specifically exemplified above, it is not intended that the invention be limited to such embodiments. Various modifications may be made thereto without departing from the scope and spirit of the present invention.

### REFERENCES

1. Rychik J. Management of protein-losing enteropathy after the Fontan procedure. Seminars in Thoracic and Cardiovascular Surgery: Pediatric Cardiac Surgery Annual 1998; 1:15-21
2. Gui-Yang S, Rychik J. Relation of mesenteric vascular resistance after Fontan operation and protein-losing enteropathy. Am J Cardiol. 2002 Sep. 15; 90(6):672-4.
3. Schulze-Neick I, Harenstein P, Li J, Stiller B, Nagdyman N, Hübler M, Butrous G, Petros A, Lange P, Redington A N. Intravenous Sildenafil is a Potent Pulmonary Vasodilator in Children with Congenital Heart Disease. Circulation. 2003; 108[suppl II]:II-167-II-173.
4. Buvinic S, Huidobro-Toro, JP. Basal tonic release of nitric oxide coupled to cGMP production regulates the vascular reactivity of the mesenteric bed. European Journal of Pharmacology 2001; 424:221-227.
5. Sharma R, Coats A J, Anker S D. The role of inflammatory mediators in chronic heart failure: cytokines, nitric oxide, and endothelin-1. Int J Cardiol. 2000 Jan. 15; 72(2): 175-86.

1. A method for treating a pediatric patient in need of improved cardiac performance comprising the administration of sildenafil, said method being effective to improve cardiac function.

2. The method of claim 1, wherein said patient is a Fontan patient.

3. The method of claim 1, wherein said patient has protein losing enteropathy.

4. The method of claim 3, wherein said patient is a Fontan patient.

5. The method of claim 1, further comprising assessing said patient for clinical improvement as demonstrated by at least one parameter selected from the group consisting of a decrease in alpha trypsin clearance, an increase in serum albumin, an increase in cardiac output, a decrease in MVR, and a reduction in circulating TNF-alpha levels.

6. A combination method for treatment of the Fontan pediatric patient comprising the administration of an effective amount of
i) an agent selected from the group consisting of of sildenafil, vardenafil HCl, or tadalafil and
ii) an agent selected from the group consisting of statins, ACE inhibitors or diuretics, said method being effective to improve cardiac function.

7. The method of claim 6, wherein said statin is selected from the group consisting of lipitor and crestor.

8. The method of claim 6, wherein said ACE inhibitor is enalapril.

9. The method of claim 6, wherein said diuretic is aldactone.

10. The method of claim 6, further comprising assessing said patient for clinical improvement as demonstrated by at least one parameter selected from the group consisting of a decrease in alpha trypsin clearance, an increase in serum albumin, an increase in cardiac output, a decrease in MVR, and a reduction in circulating TNF-alpha levels.
